(19) United States
(12) Patent Application Publication
(10) Pub. No.: US 2007/0161813 A1
(43) Pub. Date: Jul. 12, 2007

(54) Process for preparing nitriles and isonitriles by dehydration reactions with propanephosphonic anhydrides

(76) Inventors: Andreas Meudt, Hofheim (DE); Stefan Scherer, Griesheim (DE); Sven Nerdinger, Kiefersfelden (DE)

Correspondence Address: PROPAT, L.L.C., 425-C SOUTH SHARON AMITY ROAD, CHARLOTTE, NC, 28211-2841, US

(21) Appl. No.: 10/586,768

(22) PCT Filed: Jan. 15, 2005
(86) PCT No.: PCT/EP05/00361

§ 371 Date: Jul. 20, 2006

(30) Foreign Application Priority Data
Jan. 26, 2004 (DE) 10 2004 003 953.4

(51) Int. Cl. C07C 253/20

(52) U.S. Cl. 558/382

(57) ABSTRACT

The invention concerns a method for producing: a) nitriles of formula (II) and b) isonitriles of formula (III) by reacting: a) carboxylic acid amides (RCO—NH2), ammonium salts of carboxylic acids (RCOO—NH4+) or carboxylic acids in the presence of ammonia or ammonium salts (RCOOH+NH3, RCOOH+NH4+) or b) formamides (H—CO—NHR) or mixtures of amines with formic acid, with cyclic phosphonic acid anhydrides while eliminating water at a temperature ranging from −30 to +120° C., in which R represents an arbitrarily substituted linear or branched C₁-C₈ alkyl radical, a C₃-C₁₀ cycloalkyl radical, alkenyl radical, alkynyl radical or an aryl radical or heteroaryl radical. As a cyclic phosphonic acid anhydride, a 2,4,6,-substituted 1,3,5,2,4,6 -trioxatriphosphinane-2,4,6-trioxide of formula (I) is advantageously used, in which: x=3, 4 or 5; R′, independent of one another, represents open-chain or branched, saturated or unsaturated, straight-chain C₁to C₁₆ alkyl radicals or cyclic C₃ to C₁₆ alkyl radicals or aryl or heteroaryl.

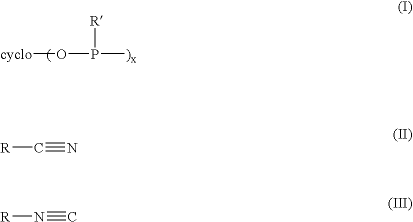

Nitriles and isonitriles are significant and extremely versatile intermediates in organic synthesis. Both compound classes exhibit a high reactivity of the C—N multiple bond, as a result of which numerous heterocarbonyl reactions are enabled. The significance in modern organic synthesis is restricted only by limitations in the availability of these compound classes. Standard processes for preparing nitriles are dehydrations of carboxamides, for which numerous reagents, for example POCl3, can be used. Isonitriles are obtainable analogously by dehydration of formamides with POCl3, and may find use, for example, in Ugi multicomponent reactions.

In modern organic synthesis, the significance of chemo-, regio- and stereoselective reagents is increasing explosively. When, for example, the intention is to convert a specific acid group to an amide in a complex molecule with numerous functional groups, some of them having only slight differences in reactivity, without influencing other groups (for example epimerization of chiral functionalities), unselective methods such as SOCl2 can no longer be used. A highly selective reagent for extremely selective amide bond formation, which affords outstanding selectivities and yields without epimerization even in oligopeptides, is propanephosphonic anhydride (T3P®). This reagent is commercially available in various solvents and conveniently usable. The performance of this reagent is so high that the problem of forming amide and peptide bonds in complex molecules can today be considered to have been solved.

There has to date been a lack of a comparable solution to the problem of converting carboxylic acids, ammonium salts of carboxylic acids and carboxamides to the corresponding nitriles and of converting formamides to the corresponding isonitriles. Although the known reagents can accomplish the desired transformations, other moieties are often likewise influenced. In many cases, the drastic conditions required epimerize even stereocenters far away.

It would therefore be very desirable to have a process which can convert carboxylic acids, ammonium salts of carboxylic acids and carboxamides by dehydration to the corresponding nitriles and formamides by dehydration to the corresponding isonitriles, but at the same time has very high chemoselectivities and is additionally usable in economically utilizable processes. The known reagents do not solve this problem, as will be demonstrated by a few examples: although POCl3 in combination with bases can accomplish the reactions mentioned, almost any possible functional group likewise reacts with this reagent. It is likewise possible to carry out the desired transformation in nitriles with dicyclohexylcarbodiimide (DCC), but partial epimerizations frequently occur at the same time; what are even worse, though, are frequently the properties of the dicyclohexylurea which is formed as a subsequent product and can barely be removed, or can only be removed by chromatographic separations, from the product. The use of water-soluble DCC derivatives is usually not performable economically as a result of their very high cost and difficult obtainability.

It has been found that, surprisingly, cyclic 2,4,6-substituted 1,3,5,2,4,6-trioxatriphosphinane 2,4,6-trioxides solve all of these problems and are an ideal and highly selective reagent for converting carboxylic acids, ammonium salts of carboxylic acids and carboxamides by dehydration to the corresponding nitriles and formamides by dehydration to the corresponding isonitriles, the desired freedom from epimerization and maximum regio- and stereoselectivity being observed at the same time.

The present invention thus relates to a highly selective process for preparing a) nitriles of the formula (II) and b) isonitriles of the formula (III)
R—C≡N (II)
R—N≡C (III)
by reacting
a) carboxamides (RCO—NH2), ammonium salts of carboxylic acids (RCOO—NH4+) or carboxylic acids in the presence of ammonia or ammonium salts (RCOOH+NH3, RCOOH+NH4+) or
b) formamides (H—CO—NHR) or mixtures of amines with formic acid, with cyclic phosphonic anhydrides with elimination of water at a temperature in the range from −30 to +120° C.,
where R may have any substitution and is a linear or branched C₁-C₈-alkyl radical, a C₃-C₁₀-cycloalkyl, alkenyl, alkynyl or an aryl or heteroaryl radical.

In a preferred inventive embodiment, the cyclic phosphonic anhydride is a 2,4,6-substituted 1,3,5,2,4,6-trioxatriphosphinane 2,4,6-trioxide of the formula (I) 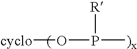
where x=3, 4 or 5,
and R′ are each independently open-chain or branched, saturated or unsaturated, straight-chain C₁ to C₁₆-alkyl radicals, in particular a C₂ to C₁₂-alkyl radical, or cyclic C₃ to C₁₆-alkyl radicals, or aryl or heteroaryl. Particular preference is given to phosphonic anhydrides of the formula (I) in which R′ is a methyl, ethyl, n-propyl, isopropyl, n-butyl, 2-butyl, isobutyl, pentyl, hexyl, in particular an ethyl, propyl, and/or butyl radical, most preferably propanephosphonic anhydride (T3P).

The dehydration to nitriles (II) and isonitriles (III) can generally be carried out at temperatures in the range from −30 to +120° C., preference being given to temperatures in the range from +30 to +70° C., lower temperatures generally correlating with higher selectivities. The reaction time is dependent upon the temperature employed and is generally from 1 to 12 hours, in particular from 3 to 6 hours.

The cyclic phosphonic anhydride can be added to the reaction medium either as a melt or as a liquid mixture dissolved in a solvent.

Suitable solvents are those which do not give rise to any side reactions with the phosphonic anhydride; these are all aprotic organic solvents, for example ligroin, butane, pentane, hexane, heptane, octane, cyclopentane, cyclohexane, cycloheptane, cyclooctane, dichloromethane, chloroform, carbon tetrachloride, 1,2-dichloroethane, 1,1,2,2-tetrachloroethane, methyl acetate, ethyl acetate, propyl acetate, butyl acetate, dimethylformamide, diethylformamide, dimethylacetamide, diethylacetamide, diethyl ether, diisopropyl ether, tert-butyl methyl ether, THF, dioxane, acetonitrile or mixtures thereof; particular preference is given to dichloromethane, chloroform, ethyl acetate, propyl acetate, butyl acetate, dimethylformamide, diethylformamide, dimethylacetamide, diethylacetamide, diisopropyl ether, tert-butyl methyl ether, THF, dioxane, acetonitrile or mixtures thereof; very particular preference is given to dichloromethane, chloroform, ethyl acetate, butyl acetate, dimethylformamide, dimethylacetamide, tert-butyl methyl ether, THF, dioxane, acetonitrile or mixtures thereof; special preference is given to THF, ethyl acetate or butyl acetate.

The phosphonic anhydride is added at least stoichiometrically in relation to the starting compound, but may also be added superstoichiometrically, for example in a ratio of 1:2.

The-reactions are preferably carried out in such a way that the appropriate amide or formamide is initially charged in a solvent, then heated to the reaction temperature, and subsequently converted to the desired nitrile or isonitrile by metering in the phosphonic anhydride as a melt or solution in one of the aforementioned solvents.

The reaction product is isolated preferably by hydrolysis and simple phase separation, since the subsequent products of the phosphonic anhydrides are generally very highly water-soluble. Depending on the nature of the product to be isolated, post-extractions may also be required. The subsequent phosphonic anhydride product formed often does not disrupt subsequent reactions, so that even the direct use of the reaction solutions obtained often brings very good results.

When an amine is to be converted to an isonitrile, this can be done very elegantly by reaction of the amine (H—CO—NHR) with formic acid and the phosphonic anhydride, which first forms the formamide which is finally dehydrated to the isonitrile. It is likewise possible to convert an amine by prior art processes first to formamides (for example with formic esters) and then to react them with the cyclic phosphonic anhydrides to give isonitriles.

When an ammonium salt of a carboxylic acid (RCOO—NH4+) is to be converted to a nitrile, this can be carried out by simple heating with the phosphonic anhydride analogously to the above-described process. This also gives rise to a very elegant and likewise extremely selective process for directly converting carboxylic acids to nitriles, by adding any ammonium salt, preferably ammonium chloride or ammonium sulfate, to the carboxylic acid and then reacting them with a phosphonic anhydride in the presence of a base.

Suitable bases are, for example, tertiary amines such as triethylamine, tripropylamine, benzyldimethylamine, N,N-dimethylaniline or pyridine. The base is added typically in a ratio of from 1 to 2 equivalents, preferably from 1 to 1.2 equivalents, based on the carboxylic acid.

The process may also be carried out in such a way that a solution or suspension of the carboxylic acid to be converted in hydrocarbons or esters such as ethyl acetate or butyl acetate is saturated with at least one equivalent of ammonia gas and subsequently treated with the phosphonic anhydride.

All procedures mentioned feature very good yields (typically 90-100%, in particular >95%) in the simultaneous absence of side reactions and of epimerizations. The selectivities of the inventive reaction are often in the range of 97-100%, in particular 99-100%.

The process according to the invention will be illustrated by the examples which follow without restricting the invention thereto:

### EXAMPLE 1

### Dehydration of Benzamide to Benzonitrile

1 mol of benzamide is initially charged in 150 ml of ethyl acetate and heated to 45° C. 1.2 mol of T3P solution in ethyl acetate (50% w/w) are metered in over the course of one hour, then the mixture is stirred at this temperature for a further three hours. At this time, the reaction GC indicates a conversion of 100%. After cooling to room temperature, 180 ml of water were added and the phases were separated. The organic phase was distilled off. The isolated yield of this reaction was 96%.

### EXAMPLE 2

### Dehydration of N-formyl-o-tolylamine to o-tolylisonitrile

0.1 mol of N-formyl-o-tolylamine is initially charged in 50 ml of ethyl acetate and heated to 55° C. 0.12 mol of T3P solution in ethyl acetate (50% w/w) is metered in over the course of one hour, then the mixture is stirred at this temperature for a further two hours. At this time, the reaction GC indicates a conversion of >99%. After cooling to 0° C., 25 ml of water were added and the phases were separated. After the solvent had been condensed out, the isonitrile remained in a yield of 97%, HPLC purity 98% (a/a).

### EXAMPLE 3

### Dehydration of Ammonium Benzoate

1 mol of ammonium benzoate is initially charged in 180 ml of butyl acetate and heated to 45° C. 1.2 mol of T3P solution in butyl acetate (50% w/w) are metered in over the course of one hour, then the mixture is stirred at this temperature for a further six hours. At this time, the reaction GC indicates a conversion of >99%. After cooling to room temperature, 140 ml of water were added and the phases were separated. The organic phase was distilled. The isolated yield of this reaction was 94%.

### EXAMPLE 4

### Benzonitrile from Benzoic Acid

1 mol of benzoic acid, 1.05 mol of ammonium chloride and 1.08 mol of triethylamine are suspended in 100 ml of ethyl acetate-and-heated to 65° C. 1.2 mol of T3P solution in ethyl acetate (50% w/w) are metered in over the course of one hour, then the mixture is stirred at this temperature for a further three hours. At this time, the reaction GC indicated a conversion of 99.8%. After cooling to room temperature, 150 ml of water were added and the phases were separated. The organic phase was distilled. The isolated yield of this reaction was 94%.

### EXAMPLE 5

### Dehydration of N-formyl-L-phenylalanine methyl ester

1 mmol of N-formyl-L-phenylalanine methyl ester is initially charged in 10 ml of ethyl acetate and heated to 28° C. 1.1 mmol of T3P solution in ethyl acetate (50% w/w) are metered in over the course of one hour, then the mixture is stirred at this temperature for a further three hours. At this time, the reaction HPLC indicated the complete consumption of the reactant. After cooling to 0° C., 5 ml of water were added and the phases were separated. After cautiously condensing out the solvent at max. 30° C., the desired chiral isonitrile remained as a colorless oil, crude yield 99%. The reaction of the isonitrile should take place at once, since rapid racemization otherwise occurs.

1. A process for preparing a) nitrites of the formula (II) and
b) isonitriles of the formula (III)
R—C≡N (II)
R—N≡C (III)
said process comprising reacting
a) carboxamides (RCO—NH2), ammonium salts of carboxylic acids (RCOO—NH4+) or carboxylic acids in the presence of ammonia or ammonium salts (RCOOH+NH3, RCOOH+NH4+) or
b) formamides (H—CO—NHR) or mixtures of amines with formic acid, with cyclic phosphonic anhydrides with elimination of water at a temperature in the range from −30 to +120° C.,
where R may have any substitution and is a linear or branched C₁-C₈-alkyl radical, a C₃-C₁₀-cycloalkyl, alkenyl, alkynyl or an aryl or heteroaryl radical.

2. The process as claimed in claim 1, wherein the cyclic phosphonic anhydride is a 2,4,6-substituted 1,3,5,2,4,6-trioxatriphosphinane 2,4,6-trioxide of the formula (I) 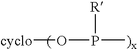
where x=3, 4 or 5 and
R′ are each independently open-chain or branched, saturated or unsaturated, straight-chain C₁ to C₁₆-alkyl radicals or cyclic C₃ to C₁₆-alkyl radicals, or aryl or heteroaryl.

3. The process as claimed in claim 2, wherein R′ is a methyl, ethyl, n-propyl, isopropyl, n-butyl, 2-butyl, isobutyl, pentyl, and/or hexyl, radical.

4. The process as claimed in claim 2, wherein the cyclic phosphonic anhydride is propanephosphonic anhydride.

5. The process as claimed in claim 1, wherein the cyclic phosphonic anhydride is either a melt or dissolved in a solvent.

6. The process as claimed in claim 5, wherein the cyclic phosphonic anhydride is in an aprotic solvent.

7. The process as claimed in claim 1, wherein said process further comprises
(i) forming a reaction solution comprising carboxamides; ammonium salts of carboxylic acids; carboxylic acids in the presence of ammonia or ammonium salts; formamide; or mixtures of amines with formic acid;
(ii) adding cyclic phosphonic anhydride to the reaction solution; and
(iii) heating the reaction solution to reaction temperature,
wherein the reaction solution is heated to the reaction temperature after addition of the phosphonic anhydride.

8. The process as claimed in claim 1, wherein nitriles are prepared and an ammonium salt together with a carboxylic acid (R—COOH) is reacted with the phosphonic anhydride in the presence of a base.

9. The process as claimed in claim 8, wherein the base is triethylamine, tripropylamine, benzyldimethylamine, N,N-dimethylaniline or pyridine.

10. The process as claimed in claim 2, wherein R′ is an ethyl, propyl, and/or butyl radical.

11. The process as claimed in claim 6, wherein the cyclic phosphonic anhydride and aprotic solvent are in a ratio of from 1:1 to 1:2.
